SEQUENCE LISTING (1 sequence)

SEQ ID NO: 1 - DNA, 24 bp, Artificial Sequence
  oligonucleotide containing unmethylated CpG motifs
tcgtcgttttgtcgttttgtcgtt

(19) United States
(12) Patent Application Publication
(10) Pub. No.: US 2006/0270707 A1
(43) Pub. Date: Nov. 30, 2006

(54) Methods and compositions using 4-[(cyclopropanecarbonylamino)methyl]-2-(2,6-dioxopiperidin-3-yl)isoindole-1,3-dione for the treatment or prevention of cutaneous lupus

(76) Inventors: Jerome B. Zeldis, Princeton, NJ (US); Patricia E.W. Rohane, Florham Park, NJ (US); Peter H. Schafer, Somerset, NJ (US)

Correspondence Address: JONES DAY, 222 EAST 41ST ST, NEW YORK, NY, 10017, US

(21) Appl. No.: 11/435,089

(22) Filed: May 16, 2006

Related U.S. Application Data
(60) Provisional application No. 60/684,499, filed on May 24, 2005.

(51) Int. Cl. A61K 31/454

(52) U.S. Cl. 514/323

(57) ABSTRACT

Methods of treating, managing or preventing cutaneous lupus are disclosed. Specific methods encompass the administration of 4-[(cyclopropanecarbonylamino)methyl]-2-(2,6-dioxopiperidin-3-yl)isoindole-1,3-dione alone or in combination with a second active agent. Pharmaceutical compositions and single unit dosage forms are also disclosed.

### 1. CROSS REFERENCE TO RELATED APPLICATIONS

This application claims the benefit under 35 U.S.C. §119 of U.S. Provisional Patent Application Ser. No. 60/684,499, filed May 24, 2005, which is incorporated herein by reference in its entirety.

### 2. FIELD OF THE INVENTION

This invention provides methods of treating, preventing and/or managing cutaneous lupus by the administration of 4-[(cyclopropanecarbonylamino)methyl]-2-(2,6-dioxopiperidin-3-yl)isoindole-1,3-dione alone or in combination with other therapeutics. The invention also provides pharmaceutical compositions and dosage forms comprising 4-[(cyclopropanecarbonylamino)methyl]-2-(2,6-dioxopiperidin-3-yl)isoindole-1,3-dione alone or in combination with other therapeutics for use in methods of treating, preventing and/or managing cutaneous lupus.

### 3. BACKGROUND OF THE INVENTION

### 3.1 Cutaneous Lupus

Lupus or lupus erythematosus is an autoimmune disorder that can cause chronic inflammation in various parts of the body, especially the skin, joints, blood, and kidneys. The body's immune system normally makes proteins called antibodies to protect the body against viruses, bacteria, and other foreign materials (i.e., antigens). In an autoimmune disorder such as lupus, the immune system loses its ability to tell the differences between antigens and its own cells and tissues and can make antibodies directed against its own cells and tissues to form immune complexes. These immune complexes can build up in the tissues and cause inflammation, injury to tissues and/or pain. The three most common types of lupus include systemic lupus erythematosus (SLE), cutaneous lupus erythematosus (CLE) and drug-induced lupus. More detailed descriptions of lupus or lupus erythematosus can be found in Wallace, 2000, The Lupus Book: A Guide for Patients and Their Families, Oxford University Press, Revised and Expanded Edition, which is incorporated by reference herein in its entirety.

Systemic lupus erythematosus (SLE) is an autoimmune disease involving multiple organ systems that is defined clinically and associated with antibodies directed against cell nuclei. SLE can affect any system or organ in the body including the joints, skin, lungs, heart, blood, kidney, or nervous system. Symptoms of SLE can range from being a minor inconvenience to very serious and even life threatening. For example, a SLE patient may experience (a) no pain or extreme pain, especially in the joints; (b) no skin manifestations or disfiguring rashes; and/or (c) no organ involvement or extreme organ damage. As discussed above, many clinical manifestations of SLE are caused by the effects of immune complexes on various tissues or cell surface components. However, it is still unclear whether polyclonal B-cell activation or a response to specific antigens exists. Nonetheless, a genetic predisposition to the development of SLE may exist. More detailed descriptions of SLE can be found in Lahita, 1999, Systemic Lupus Erythematosus, Academic Press, Third Edition, which is incorporated by reference herein in its entirety.

Drug-induced lupus generally occurs after the use of certain prescribed drugs. The symptoms of drug-induced lupus are similar to those of SLE. The drugs most commonly connected with drug-induced lupus are hydralazine (used to treat high blood pressure or hypertension) and procainamide (used to treat irregular heart rhythms). However, only an extremely small number who take these drugs can develop overt drug-induced lupus. The symptoms usually fade when the medications are discontinued.

Cutaneous lupus or cutaneous lupus erythematosus affects primarily the skin and is generally characterized by skin inflammation, skin rashes and hemorrhages in the skin. Cutaneous lupus may also affect hair and mucous membranes but usually does not involve internal organs like SLE. Cutaneous lupus can be categorized into groups including acute cutaneous lupus erythematosus (ACLE), subacute cutaneous lupus erythematosus (SCLE), chronic cutaneous lupus erythematosus (CCLE) or discoid lupus erythematosus (DLE) and neonatal lupus erythematosus (NLE). More detailed descriptions of cutaneous lupus or cutaneous lupus erythematosus can be found in Kuhn et al., 2004, Cutaneous Lupus Erythematosus, Springer, First Edition, which is incorporated by reference herein in its entirety.

ACLE is generally a photosensitive dermatosis. It can appear as flattened areas of red skin that resemble a persistent sunburn or have a rash-like appearance. ACLE may erupt in a butterfly pattern localized to the central portion of the face and/or in a generalized pattern including other areas such as the arms, legs and body. The etiology of ACLE is believed to be multi-factorial, involving genetic, environmental and hormonal factors. In patients who are predisposed genetically, ACLE can be triggered by viruses (e.g., EBV) and exposure to ultraviolet light.

SCLE is a non-scarring non-atrophy-producing photosensitive dermatosis. In some cases, SCLE appears as a non-itchy ring-shaped dry rash on the upper back and chest, often following sun exposure. SCLE may occur in patients with systemic lupus erythematosus, Sjögren syndrome and deficiency of the second component of complement (C2d) or it can be drug induced. SCLE usually occurs in genetically predisposed individuals, most often in patients with human leukocyte antigen B8 (HLA-B8), human leukocyte antigen DR3 (HLA-DR3), human leukocyte antigen DRw52 (HLA-DRw52) and human leukocyte antigen DQ1 (HLA-DQ1). SCLE strongly associates with anti-Ro (SS-A) autoantibodies. Usually, SCLE manifests following UV light exposure, but other triggers or inciting factors are also implicated.

CCLE or DLE is a chronic, scarring, atrophy producing, photosensitive dermatosis. DLE commonly appears as red scaly patches which leave white scars. DLE predominantly affects the cheeks and nose, but sometimes involves the upper back, neck, backs of hands, bald areas in scalp and the lips. DLE may occur in patients with systemic lupus erythematosus (SLE). Some patients also have the lesions of SCLE and some may have a malar rash. Therapy with sunscreens, topical corticosteroids and antimalarials can be effective. DLE probably occurs in genetically predisposed individuals, but the exact genetic connection has not been determined. The pathophysiology of DLE is not well understood. It has been suggested that a heat shock protein is induced in the keratinocyte following ultraviolet (UV) light exposure or stress and this protein may act as a target for γδ T-cell-mediated epidermal cell cytotoxicity.

Verrucous DLE, lupus profundus, mucosal DLE, palmar-plantar DLE and lupus tumidus are some specific forms of DLE. Verrucous DLE refers to DLE having lesions that can develop into very thick scales. Lupus profundus refers to DLE having lesions that may occur in conjunction with firm lumps in the fatty tissue underlying the skin. Mucosal DLE refers to the lesions that occasionally occur in the mucus membranes of the mouth, nose and eyes. Palmar-plantar DLE refers to the lesions that occasionally occur on the hands and feet. Lupus tumidus appears as smooth, shiny, red-violet plaques of the head and neck that can be pruritic and have a fine scale. The lupus tumidus lesions usually clear without scarring and can recur in their original distribution.

NLE is a rare condition in children and usually appears as nonscarring, non-atrophy-producing lesions. In some cases, newborn babies born to mothers with SCLE may develop NLE with a temporary ring-like or annular rash. NLE is believed to be related to various factors including genetic predisposition, viral infection and other unknown factors. NLE may affect the skin, heart, liver, blood-forming elements or the spleen.

Lupus erythematosus (LE) of childhood relates to genetic factors and perhaps other environmental events. LE of childhood may affect the skin or it may manifest as systemic LE and affect any organ system in the body, most commonly the kidneys, joints and blood.

### 4. SUMMARY OF THE INVENTION

In one aspect, the invention provides methods of treating, preventing and/or managing cutaneous lupus. The methods comprise administering to a patient in need of such treatment, prevention or management a therapeutically or prophylactically effective amount of 4-[(cyclopropanecarbonylamino)methyl]-2-(2,6-dioxopiperidin-3-yl)isoindole-1,3-dione or a pharmaceutically acceptable salt, solvate (e.g., hydrate) or stereoisomer thereof.

In some embodiments, the methods further comprise the administeration of a therapeutically or prophylactically effective amount of at least a second active agent wherein the second active agent is an immunomodulatory compound, an anti-inflammatory such as non-steroidal agents (e.g., salicylates) or corticosteroids (e.g., dexamethasone), an anti-malarial, an immunosuppressant, an antibiotic, an antiviral, an immunoglobulin, an immunologic-enhancing drug or a hormone.

In another embodiment, the 4-[(cyclopropanecarbonylamino)methyl]-2-(2,6-dioxopiperidin-3-yl)isoindole-1,3-dione or a pharmaceutically acceptable salt, solvate or stereoisomer thereof is administered topically in a dosage form selected from the group consisting of ointments, creams, gels, pastes, dusting powders, lotions, sprays, liniments, poultices, aerosols, solutions, emulsions, suspensions and combinations thereof.

In further embodiments, the 4-[(cyclopropanecarbonylamino)methyl]-2-(2,6-dioxopiperidin-3-yl)isoindole-1,3-dione or a pharmaceutically acceptable salt, solvate or stereoisomer thereof is administered parenterally or orally or in a controlled-release manner.

In another aspect, the invention provides pharmaceutical compositions for treating, preventing and/or managing cutaneous lupus comprising 4-[(cyclopropanecarbonylamino)methyl]-2-(2,6-dioxopiperidin-3-yl)isoindole-1,3-dione or a pharmaceutically acceptable salt, solvate or stereoisomer thereof.

In some embodiments, the invention provides single unit dosage forms for treating, preventing and/or managing cutaneous lupus comprising the 4-[(cyclopropanecarbonylamino)methyl]-2-(2,6-dioxopiperidin-3-yl)isoindole-1,3-dione or a pharmaceutically acceptable salt, solvate or stereoisomer thereof.

### b 5. DETAILED DESCRIPTION OF THE INVENTION

A first aspect of the invention encompasses methods of treating, managing and/or preventing cutaneous lupus which comprises administering to a patient in need of such treatment or prevention a therapeutically or prophylactically effective amount of 4-[(cyclopropanecarbonylamino)methyl]-2-(2,6-dioxopiperidin-3-yl)isoindole-1,3-dione or a pharmaceutically acceptable salt, solvate or stereoisomer thereof.

Examples of cutaneous lupus include, but not limited to, acute cutaneous lupus erythematosus (ACLE), subacute cutaneous lupus erythematosus (SCLE), neonatal lupus erythematosus (NLE), lupus erythematosus of childhood and discoid lupus erythematosus (DLE) including verrucous DLE, lupus profundus, mucosal DLE, palmar-plantar DLE and lupus tumidus. Furthermore, the patients to be treated included mammals, particularly human. Children and adults can be treated by the methods and compositions disclosed herein. Immunocompromised patients may also be treated. This invention contemplates treatment of patients that have not used other therapies, those that that have used other therapies and those refractory to therapies for lupus such as cutaneous lupus mentioned above.

### 5.1 Definitions

As used herein and unless otherwise indicated, the term “pharmaceutically acceptable salt” includes, but is not limited to, salts of acidic or basic groups that can be present in the compounds of the invention. The compound of the invention is basic in nature and capable of forming a wide variety of salts with various inorganic and organic acids. The acids that can be used to prepare pharmaceutically acceptable salts of such basic compounds are those that form salts comprising pharmacologically acceptable anions including, but not limited to, acetate, benzenesulfonate, benzoate, bicarbonate, bitartrate, bromide, calcium edetate, camsylate, carbonate, chloride, bromide, iodide, citrate, dihydrochloride, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide and pamoate. The compound of the invention includes an amino group that can form pharmaceutically acceptable salts with various amino acids, in addition to the acids mentioned above. The compound of the invention is also acidic in nature and capable of forming base salts with various pharmacologically acceptable cations. Non-limiting examples of such salts include alkali metal or alkaline earth metal salts and, particularly, calcium, magnesium, sodium, lithium, zinc, potassium and iron salts.

As used herein and unless otherwise indicated, the term “hydrate” means a compound of the present invention or a salt thereof, that further includes a stoichiometric or non-stoichiometeric amount of water bound by non-covalent intermolecular forces.

As used herein and unless otherwise indicated, the term “solvate” means a solvate formed from the association of one or more solvent molecules to a compound of the present invention. The term “solvate” includes hydrates (e.g., mono-hydrate, dihydrate, trihydrate, tetrahydrate and the like).

As used herein and unless otherwise indicated, the term “polymorph” means solid crystalline forms of a compound of the present invention or complex thereof. Different polymorphs of the same compound can exhibit different physical, chemical and/or spectroscopic properties.

As used herein, and unless otherwise specified, the terms “biohydrolyzable carbamate,” “biohydrolyzable carbonate,” “biohydrolyzable ureide” and “biohydrolyzable phosphate” mean a carbamate, carbonate, ureide and phosphate, respectively, of a compound that either: 1) does not interfere with the biological activity of the compound but can confer upon that compound advantageous properties in vivo, such as uptake, duration of action or onset of action; or 2) is biologically inactive but is converted in vivo to the biologically active compound. Non-limiting examples of biohydrolyzable carbamates include lower alkylamines, substituted ethylenediamines, aminoacids, hydroxyalkylamines, heterocyclic and heteroaromatic amines and polyether amines.

As used herein, and unless otherwise specified, the term “stereoisomer” encompasses all enantiomerically/stereomerically pure and enantiomerically/stereomerically enriched compounds of this invention.

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure when the compound contains 80%, 90% or 95% or more of one stereoisomer and 20%, 10% or 5% or less of the counter stereoisomer. In some cases, a compound of the invention is considered optically active or stereomerically/enantiomerically pure (i.e., substantially the R-form or substantially the S-form) with respect to a chiral center when the compound is about 80% ee (enantiomeric excess) or greater, preferably, equal to or greater than 90% ee with respect to a particular chiral center and more preferably 95% ee with respect to a particular chiral center.

As used herein, and unless otherwise indicated, the term “stereomerically enriched” or “enantiomerically enriched” encompasses racemic mixtures as well as other mixtures of stereoisomers of compounds of this invention (e.g., R/S=30/70, 35/65, 40/60, 45/55, 55/45, 60/40, 65/35 and 70/30).

As used herein, and unless otherwise specified, the terms “treat,” “treating” and “treatment” contemplate an action that occurs while a patient is suffering from the specified disease or disorder, which reduces the severity or symptoms of the disease or disorder or retards or slows the progression or symptoms of the disease or disorder.

As used herein, unless otherwise specified, the terms “prevent,” “preventing” and “prevention” contemplate an action that occurs before a patient begins to suffer from the specified disease or disorder, which inhibits or reduces the severity or symptoms of the disease or disorder.

As used herein, and unless otherwise indicated, the terms “manage,” “managing” and “management” encompass preventing the recurrence of the specified disease or disorder in a patient who has already suffered from the disease or disorder and/or lengthening the time that a patient who has suffered from the disease or disorder remains in remission. The terms encompass modulating the threshold, development and/or duration of the disease or disorder or changing the way that a patient responds to the disease or disorder.

As used herein, and unless otherwise specified, the term “enhancing” or “enhance,” when used in connection with immune response, means that when an antigenic or immunogenic agent is administered to a subject who has been or is being treated with 4-[(cyclopropanecarbonylamino)methyl]-2-(2,6-dioxopiperidin-3-yl)isoindole-1,3-dione, there is an increased antibody formation, as compared to a subject to which same amount of the antigenic or immunogenic agent alone is administered, as determined by any conventional methods of antibody level determination known in the art, for example, nephelometry, immunoelectrophoresis, radioimmunoassay and ELISA. In some embodiments, when methods of this invention are used, antibody formation is increased by about 5%, 10%, 20%, 50% or 100% or more, as compared to the antibody formation obtained when such methods are not used.

### 5.2 Method of Treatments and Prevention

The present invention provides methods of treating, preventing and/or managing cutaneous lupus. Non-limiting examples of cutaneous lupus include cutaneous lupus erythematosus, subacute cutaneous lupus erythematosus, chronic cutaneous lupus erythematosus or discoid lupus erythematosus, neonatal lupus erythematosus, verrucous DLE, lupus profundus, mucosal DLE, palmar-plantar DLE and lupus tumidus.

In some embodiments, the present invention provides methods of treating ACLE. ACLE is generally a photosensitive dermatosis. It can appear as flattened areas of red skin that resemble a persistent sunburn or have a rash-like appearance. ACLE may erupt in a butterfly pattern localized to the central portion of the face and/or in a generalized pattern including other areas such as the arms, legs and body. The etiology of ACLE is believed to be multi-factorial, involving genetic, environmental and hormonal factors. In patients who are predisposed genetically, exposure to natural ultraviolet radiation is a frequent precipitating factor for ACLE.

In further embodiments, the present invention provides methods of treating SCLE. SCLE is a non-scarring non-atrophy-producing photosensitive dermatosis. In some cases, SCLE appears as a non-itchy ring-shaped dry rash on the upper back and chest, often following sun exposure. SCLE may occur in patients with systemic lupus erythematosus, Sjögren syndrome and deficiency of the second component of complement (C2d) or it can be drug induced. SCLE usually occurs in genetically predisposed individuals, most often in patients with human leukocyte antigen B8 (HLA-B8), human leukocyte antigen DR3 (HLA-DR3), human leukocyte antigen DRw52 (HLA-DRw52) and human leukocyte antigen DQ1 (HLA-DQ1). SCLE strongly associates with anti-Ro (SS-A) autoantibodies.

In further embodiments, the present invention provides methods of treating CCLE or DLE. CCLE or DLE is a chronic, scarring, atrophy producing, photosensitive dermatosis. DLE commonly appears as red scaly patches which leave white scars. DLE predominantly affects the cheeks and nose, but sometimes involves the upper back, neck, backs of hands, bald areas in scalp and the lips. DLE may occur in patients with systemic lupus erythematosus (SLE). Some patients also have the lesions of SCLE and some may have a malar rash. Therapy with sunscreens, topical corticosteroids and antimalarials can be effective. DLE probably occurs in genetically predisposed individuals, but the exact genetic connection has not been determined. The pathophysiology of DLE is not well understood. It has been suggested that a heat shock protein is induced in the keratinocyte following ultraviolet (UV) light exposure or stress and this protein may act as a target for γδ T-cell-mediated epidermal cell cytotoxicity.

In further embodiments, the present invention provides methods of treating verrucous DLE. Verrucous DLE is a specific form of DLE and refers to DLE having lesions that can develop into very thick scales.

In further embodiments, the present invention provides methods of treating lupus profundus. Lupus profundus is a specific form of DLE and refers to DLE having lesions that may occur in conjunction with firm lumps in the fatty tissue underlying the skin.

In further embodiments, the present invention provides methods of treating mucosal DLE. Mucosal DLE is a specific form of DLE and refers to the lesions that occasionally occur in the mucus membranes of the mouth, nose and eyes.

In further embodiments, the present invention provides methods of treating palmar-plantar DLE. Palmar-plantar DLE is a specific form of DLE and refers to the lesions that occasionally occur on the hands and feet.

In further embodiments, the present invention provides methods of treating lupus tumidus. Lupus tumidus is a specific form of DLE and appears as smooth, shiny, red-violet plaques of the head and neck that can be pruritic and have a fine scale. The lupus tumidus lesions usually clear without scarring and can recur in their original distribution.

In further embodiments, the present invention provides methods of treating NLE. NLE is a rare condition in children and usually appears as nonscarring, non-atrophy-producing lesions. In some cases, newborn babies born to mothers with SCLE may develop NLE with a temporary ring-like or annular rash. NLE is believed to be related to various factors including genetic predisposition, viral infection and other unknown factors. NLE may affect the skin, heart, liver, blood-forming elements or the spleen.

In further embodiments, the present invention provides methods of treating Lupus erythematosus (LE) of childhood. Lupus erythematosus (LE) of childhood relates to genetic factors and perhaps other environmental events. LE of childhood may affect the skin or it may manifest as systemic LE and affect any organ system in the body, most commonly the kidneys, joints and blood.

Methods encompassed by this invention comprise administering 4-[(cyclopropanecarbonylamino)methyl]-2-(2,6-dioxopiperidin-3-yl)isoindole-1,3-dione or a pharmaceutically acceptable salt, solvate or stereoisomer thereof, to a patient (e.g., a human) suffering or likely to suffer, from cutaneous lupus as described herein.

This invention also encompasses the uses of 4-[(cyclopropanecarbonylamino)methyl]-2-(2,6-dioxopiperidin-3-yl)isoindole-1,3-dione in modulating the immune system to keep it from slipping into imbalance and producing inflammatory and autoimmune disorders like lupus in a patient. Therefore, in another embodiment, this invention encompasses methods of enhancing an immune response to an immunogen, comprising administering a therapeutically or prophylactically effective amount of 4-[(cyclopropanecarbonylamino)methyl]-2-(2,6-dioxopiperidin-3-yl)isoindole-1,3-dione or a pharmaceutically acceptable salt, solvate or stereoisomer thereof, to a patient in need of such enhancement. 4-[(Cyclopropanecarbonylamino)methyl]-2-(2,6-dioxopiperidin-3-yl)isoindole-1,3-dione can be administered prior to, during or subsequent to the patient's exposure to the immunogen.

In some embodiments, the patient is a cutaneous lupus patient. In other embodiments, the patient is a female. In further embodiments, the patient is a male. In further embodiments, the patient is a child.

In a further embodiment, 4-[(cyclopropanecarbonylamino)methyl]-2-(2,6-dioxopiperidin-3-yl)isoindole-1,3-dione can be used to enhance the effect of vaccines such as, but not limited to, vaccines for pathogenic disorders, cancer and autoimmune diseases. This invention also encompasses a method of enhancing the immune response to an immunogen in a patient, comprising administering to a patient in need of such enhancement 4-[(cyclopropanecarbonylamino)methyl]-2-(2,6-dioxopiperidin-3-yl)isoindole-1,3-dione and a vaccine comprising the immunogen. 4-[(Cyclopropanecarbonylamino)methyl]-2-(2,6-dioxopiperidin-3-yl)isoindole-1,3-dione can be administered prior to, simultaneously with and subsequent to the administration of the vaccine. In some embodiments, the patient is a cutaneous lupus patient. In other embodiments, the patient is a female. In further embodiments, the patient is a male. In further embodiments, the patient is a child. In further embodiments, the patient is under 18 years old.

Non-limiting examples of suitable vaccines that can be used with 4-[(cyclopropanecarbonylamino)methyl]-2-(2,6-dioxopiperidin-3-yl)isoindole-1,3-dione include those comprising antigens from an animal, a plant, a bacteria, a protozoan, a parasite, a virus or a combination thereof. The antigenic or immunogenic agent can be any viral peptide, protein, polypeptide or a fragment thereof derived from a virus including, but not limited to, RSV-viral proteins, e.g., RSV F glycoprotein, RSV G glycoprotein, influenza viral proteins, e.g., influenza virus neuraminidase, influenza virus hemagglutinin, herpes simplex viral protein, e.g., herpes simplex virus glycoprotein including for example, gB, gC, gD and gE. The antigenic or immunogenic agent for use in the compositions of the invention can be an antigen of a pathogenic virus such as, an antigen of adenovirdiae (e.g., mastadenovirus and aviadenovirus), herpesviridae (e.g., herpes simplex virus 1, herpes simplex virus 2, herpes simplex virus 5 and herpes simplex virus 6), leviviridae (e.g., levivirus, enterobacteria phase MS2, allolevirus), poxviridae (e.g., chordopoxvirinae, parapoxvirus, avipoxvirus, capripoxvirus, leporipoxvirus, suipoxvirus, molluscipoxvirus and entomopoxvirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), metapneumovirus (e.g., avian pneumovirus and human metapneumovirus), picornaviridae (e.g., enterovirus, rhinovirus, hepatovirus (e.g., human hepatitis A virus), cardiovirus and apthovirus), reoviridae (e.g., orthoreovirus, orbivirus, rotavirus, cypovirus, fijivirus, phytoreovirus and oryzavirus), retroviridae (e.g., mammalian type B retroviruses, mammalian type C retroviruses, avian type C retroviruses, type D retrovirus group, BLV-HTLV retroviruses), lentivirus (e.g. human immunodeficiency virus 1 and human immunodeficiency virus 2), spumavirus, flaviviridae (e.g., hepatitis C virus), hepadnaviridae (e.g., hepatitis B virus), togaviridae (e.g., alphavirus (e.g., sindbis virus) and rubivirus (e.g., rubella virus), rhabdoviridae (e.g., vesiculovirus, lyssavirus, ephemerovirus, cytorhabdovirus and necleorhabdovirus), arenaviridae (e.g., arenavirus, lymphocytic choriomeningitis virus, Ippy virus and lassa virus) and coronaviridae (e.g., coronavirus and torovirus).

The immunogen for use in the vaccine can be any substance that under appropriate conditions results in an immune response in a subject, including, but not limited to, polypeptides, peptides, proteins, glycoproteins, lipids, nucleic acids and polysaccharides. The concentration of immunogen in the vaccine can be determined using standard methods known to one skilled in the art and depends on the potency and nature of the immunogen.

Patients in need of humoral immunity boost can be determined based on variety of factors, including, but not limited to, demographics, genetic factors and work environment. Persons who dwell in or travel to an area where high level exposure to pathogens is likely are one example of such patients. Persons who have family history of genetically descended immune disorder are another example. Furthermore, persons who are typically exposed to high level of pathogens (e.g., health workers) are yet another example of such patients.

### 5.2.1 Combination Therapy with a Second Active Agent of Therapy

In particular methods encompassed by this embodiment, 4-[(cyclopropanecarbonylamino)methyl]-2-(2,6-dioxopiperidin-3-yl)isoindole-1,3-dione is administered in combination with another drug (“second active agent”) or method of treating, managing and/or preventing cutaneous lupus. Second active agents include small molecules and large molecules (e.g., proteins and antibodies), examples of which are provided herein, as well as stem cells. Non-limiting examples of methods or therapies that can be used in combination with the administration of 4-[(cyclopropanecarbonylamino)methyl]-2-(2,6-dioxopiperidin-3-yl)isoindole-1,3-dione include antibody injections or infusions and stem cell transplantation.

4-[(Cyclopropanecarbonylamino)methyl]-2-(2,6-dioxopiperidin-3-yl)isoindole-1,3-dione can be combined with at least a second active agent in methods of the invention. This invention encompasses synergistic combinations for the treatment, prevention and/or management of cutaneous lupus. 4-[(Cyclopropanecarbonylamino)methyl]-2-(2,6-dioxopiperidin-3-yl)isoindole-1,3-dione can also be used to alleviate adverse or unnamed effects associated with some second active agents and conversely some second active agents can be used to alleviate adverse or unnamed effects associated with 4-[(cyclopropanecarbonylamino)methyl]-2-(2,6-dioxopiperidin-3-yl)isoindole-1,3-dione.

One or more second active agents can be used in the methods of the invention together with 4-[(cyclopropanecarbonylamino)methyl]-2-(2,6-dioxopiperidin-3-yl)isoindole-1,3-dione. The second active agent can be a large molecule (e.g., tharapeutic peptides, proteins, antibodies, antigens or vaccines) or a small molecule (e.g., inorganic, organometallic or organic molecules having a molecular weight below 1000). This invention also encompasses the use of native, naturally occurring and recombinant proteins.

This invention encompasses the uses of second active agents that are small molecules to alleviate adverse and unnamed effects that might associate with the administration of 4-[(cyclopropanecarbonylamino)methyl]-2-(2,6-dioxopiperidin-3-yl)isoindole-1,3-dione. This invention encompasses synergistic combinations 4-[(cyclopropanecarbonylamino)methyl]-2-(2,6-dioxopiperidin-3-yl)isoindole-1,3-dione with small second active agents for the treatment, prevention and/or management of cutaneous lupus. The second active agents can be administered before, after or simultaneously with 4-[(cyclopropanecarbonylamino)methyl]-2-(2,6-dioxopiperidin-3-yl)isoindole-1,3-dione.

In some embodiments of interest, the second active agents may include, but are not limited to, anti-inflammatories such as, but not limited to, acetaminophen (e.g., TYLENOL®) 5-aminosalicylic acid derivatives, salicylates, corticosteroids and nonsteroidal anti-inflammatory drugs. A non-limiting example of 5-aminosalicylic acid derivatives is sulfasalazine (e.g., AZULFIDINE®). A non-limiting examples of salicylates is acetylsalicylic acid (e.g., ASPIRIN®).

Non-limiting examples of corticosteroids include dexamethasone (e.g., AZIUM® or VOREN®), hydrocortisone (e.g., CETACORT®, HYTONE® or NUTRACORT®), beclomethasone (e.g., VANCERIL®), budesonide (e.g., PULMICORT®), fluticasone (e.g., FLONASE® or FLOVENT®), methylprednisolone (e.g., DEPO-MEDROL®, SOLU-MEDROL® or MEDROL®), mometasone furoate (e.g., NASONE® or ELOCON®), prednisone (e.g., DELTASON®, ORASON®, PREDNICEN-M® or LIQUID PRED®) and triamcinolone (e.g., AZMACORT®).

Non-limiting examples of corticosteroids include diclofenac (e.g., ARTHROTEC®), diflunisal (e.g., DOLOBID®), etodolac (e.g., LODINE®) fenoprofen (e.g., NALFON®), ibuprofen (e.g., ADVIL, CHILDREN'S ADVIL/MOTRIN, MEDIPREN, MOTRIN, NUPRIN or PEDIACARE FEVER®), indomethacin (e.g., ARTHREXIN®), ketoprofen (e.g., ORUVAIL®), ketorolac (e.g., TORADOL®), fosfomycin tromethamine (e.g., MONURAL®), meclofenamate (e.g., Meclomen®), nabumetone (e.g., RELAFEN®), naproxen (e.g., ANAPROX®, ANAPROX® DS, EC-NAPROSYN®, NAPRELAN® or NAPROSYN®), oxaprozin (e.g., DAYPRO®), piroxicam (e.g., FELDENE®), sulindac (e.g., CLINORIL®), and tolmetin (e.g., TOLECTIN® DS or TOLECTIN®).

In other embodiments of interest, the second active agents may include, but are not limited to, anti-malarials such as chloroquine (e.g., ARALEN®) and hydroxychloroquine (e.g., PLAQUENIL®); immunosuppressants such as azathioprine (e.g., IMURAN®^{M}), cyclophosphamide (e.g., CYTOXAN®), chlorambucil (e.g., LEUKERAN®) and melphalan (e.g., ALKERAN®); and immunomodulatory compounds such as thalidomide (e.g., THALOMID®), azathioprine (e.g., IMURAN®), cyclophosphamide (e.g., CYTOXAN®), methotrexate (e.g., RHEUMATREX®) and cyclosporin (e.g., NEORAL® or SANDIMMUNE®).

In further embodiments of interest, the second active agents may include, but are not limited to, antibiotics (therapeutic or prophylactic) such as, but not limited to, ampicillin (e.g., UNASYN®), tetracycline (e.g., ACHROMYCIN® or SUMYCIN®), penicillin (e.g., AMOXIL®, POLYMOX®, TRIMOX®, SPECTROBID® or GEOCILLIN®), cephalosporins (e.g., OMNICEF®, SPECTRACEF®, SUPRAX®, VANTIN®, CEFZIL® or CEDAX®), streptomycin (e.g., ZANOSAR®), kanamycin (e.g., KANTREX®) and erythromycin (e.g., E.E.S.®, E-MYCIN®, ERYC®, ERY-TAB®, ERYTHROCIN® or PCE®); antivirals such as, but not limited to, amantadine (e.g., SYMMETREL®), rimantadine (e.g., FLUMADINE®), acyclovir (e.g., ZOVIRAX®) and ribavirin (e.g., VIRAZOLE®); immunoglobulin; immunologic enhancing drugs such as, but not limited to, levamisole (e.g., ERGAMISOL®) and inosine pranobex (ISOPRINOSINE®); biologics such as, but not limited to, gammaglobulin, transfer factor, interleukins and interferons; hormones such as, but not limited to, thymic; and other immunologic agents such as, but not limited to, B cell stimulators (e.g., BAFF/BlyS), cytokines (e.g., IL-2, IL-4 and IL-5), growth factors (e.g., TGF-β), antibodies (e.g., anti-CD40 and IgM), oligonucleotides containing unmethylated CpG motifs (e.g., TCGTCGTTTTGTCGTTTTGTCGTT) and vaccines (e.g., viral and tumor peptide vaccines).

In another embodiment, methods of this invention can be used in combination with other methods used for the treatment, prevention and/or management of cutaneous lupus. Examples of other methods include, but not limited to, stem cell transplantation, enzyme replacement therapy using, for example, bovine adenosine deaminase conjugated to polyethylene glycol (PEG-ADA), fetal thymus transplant, cultured neonatal thymus transplant, thymic epithelial cell transplant and fetal liver transplant.

Specific methods of the invention comprise administering 4-[(cyclopropanecarbonylamino)methyl]-2-(2,6-dioxopiperidin-3-yl)isoindole-1,3-dione or a pharmaceutically acceptable salt, solvate or stereoisomer thereof, in combination with at least a second active agent or another therapy.

Administration of 4-[(cyclopropanecarbonylamino)methyl]-2-(2,6-dioxopiperidin-3-yl)isoindole-1,3-dione and at least a second active agent to a patient can occur simultaneously or sequentially by the same or different routes of administration. The suitability of a particular route of administration employed for a particular second active agent will depend on the second active agent itself (e.g., whether it can be administered topically or orally without decomposition prior to entering the blood stream) and the disease being treated. A particular route of administration for 4-[(cyclopropanecarbonylamino)methyl]-2-(2,6-dioxopiperidin-3-yl)isoindole-1,3-dione is topical administration. Particular routes of administration for the second active agents or ingredients of the invention are known to those of ordinary skill in the art. See, e.g., The Merck Manual, 1023-1041 (17^{th} ed., 1999).

The amount of second active agent administered can be determined based on the specific agent used, the type of disease being treated or managed, the severity and stage of disease and the amount(s) of 4-[(cyclopropanecarbonylamino)methyl]-2-(2,6-dioxopiperidin-3-yl)isoindole-1,3-dione and any optional additional second active agents concurrently administered to the patient. Those of ordinary skill in the art can determine the specific amounts according to conventional procedures known in the art. In the beginning, one can start from the amount of the second active agent that is conventionally used in the therapies and adjust the amount according to the factors described above. See, e.g., Physician's Desk Reference (56^{th} Ed., 2004).

In one embodiment of the invention, the second active agent is administered intravenously or subcutaneously and once or twice daily in an amount of from about 1 to about 1000 mg, from about 5 to about 500 mg, from about 10 to about 350 mg or from about 50 to about 200 mg. The specific amount of the second active agent will depend on the specific agent used, the type of disease being treated or managed, the severity and stage of disease and the amount(s) of 4-[(cyclopropanecarbonylamino)methyl]-2-(2,6-dioxopiperidin-3-yl)isoindole-1,3-dione and any optional additional second active agents concurrently administered to the patient. In one embodiment, 4-[(cyclopropanecarbonylamino)methyl]-2-(2,6-dioxopiperidin-3-yl)isoindole-1,3-dione can be administered in an amount of from about 0.1 to about 150 mg and preferably from about 1 to about 25 mg, more preferably from about 2 to about 10 mg orally and daily alone or in combination with a second active agent disclosed herein, prior to, during or after the use of conventional therapy.

### 5.2.2 Cycling Therapy

In some embodiments, the prophylactic or therapeutic agents of the invention can be cyclically administered to a patient. Cycling therapy involves the administration of a second active agent for a period of time, followed by a rest for a period of time and repeating this sequential administration. Cycling therapy can reduce the development of resistance to one or more of the therapies, avoid or reduce the side effects of one of the therapies and/or improves the efficacy of the treatment.

Consequently, in one specific embodiment of the invention, 4-[(cyclopropanecarbonylamino)methyl]-2-(2,6-dioxopiperidin-3-yl)isoindole-1,3-dione is administered daily in a single or divided doses in a four to six week cycle with a rest period of about a week or two weeks. The invention further allows the frequency, number and length of dosing cycles to be increased. Thus, another specific embodiment of the invention encompasses the administration of 4-[(cyclopropanecarbonylamino)methyl]-2-(2,6-dioxopiperidin-3-yl)isoindole-1,3-dione for more cycles than are typical when it is administered alone. In yet another specific embodiment of the invention, 4-[(cyclopropanecarbonylamino)methyl]-2-(2,6-dioxopiperidin-3-yl)isoindole-1,3-dione is administered for a greater number of cycles that would typically cause dose-limiting toxicity in a patient to whom a second active ingredient is not also being administered.

In one embodiment, 4-[(cyclopropanecarbonylamino)methyl]-2-(2,6-dioxopiperidin-3-yl)isoindole-1,3-dione is administered daily and continuously for three or four weeks at a dose of from about 0.1 to about 150 mg/d followed by a break of one or two weeks. In another embodiment, 4-[(cyclopropanecarbonylamino)methyl]-2-(2,6-dioxopiperidin-3-yl)isoindole-1,3-dione is administered daily and continuously at an initial dose of 0.1 to 5 mg/d with dose escalation (every week) by 1 to 10 mg/d to a maximum dose of 50 mg/d for as long as therapy is tolerated. In a particular embodiment, 4-[(cyclopropanecarbonylamino)methyl]-2-(2,6-dioxopiperidin-3-yl)isoindole-1,3-dione is administered in an amount of about 1, 5, 10 or 25 mg/day, preferably in an amount of about 10 mg/day for three to four weeks, followed by one week or two weeks of rest in a four or six week cycle.

In another embodiment of the invention, 4-[(cyclopropanecarbonylamino)methyl]-2-(2,6-dioxopiperidin-3-yl)isoindole-1,3-dione and a second active ingredient are administered orally, with administration of 4-[(cyclopropanecarbonylamino)methyl]-2-(2,6-dioxopiperidin-3-yl)isoindole-1,3-dione occurring 30 to 60 minutes prior to a second active ingredient, during a cycle of four to six weeks. In another embodiment of the invention, the combination of 4-[(cyclopropanecarbonylamino)methyl]-2-(2,6-dioxopiperidin-3-yl)isoindole-1,3-dione and a second active ingredient is administered by intravenous infusion over about 90 minutes every cycle. In a specific embodiment, one cycle comprises the administration of from about 1 to about 25 mg/day of 4-[(cyclopropanecarbonylamino)methyl]-2-(2,6-dioxopiperidin-3-yl)isoindole-1,3-dione and from about 50 to about 200 mg/m²/day of a second active ingredient daily for three to four weeks and then one or two weeks of rest. In another specific embodiment, each cycle comprises the administration of from about 5 to about 10 mg/day of 4-[(cyclopropanecarbonylamino)methyl]-2-(2,6-dioxopiperidin-3-yl)isoindole-1,3-dione and from about 50 to about 200 mg/m²/day of a second active ingredient for 3 to 4 weeks followed by one or two weeks of rest. Typically, the number of cycles during which the combinatorial treatment is administered to a patient will be from about one to about 24 cycles, more typically from about two to about 16 cycles and even more typically from about four to about three cycles.

### 5.3 4-[(CYCLOPROPANECARBONYLAMINO)METHYL]-2-(2,6-DIOXOPIPERIDIN-3-YL)ISOINDOLE-1,3-DIONE

The present invention provides methods of treating, managing or preventing cutaneous lupus, which comprises administering to a patient in need of such treatment, management or prevention a therapeutically or prophylactically effective amount of 4 [(cyclopropanecarbonylamino)methyl]-2-(2,6-dioxopiperidin-3-yl)isoindole-1,3-dione having the following formula: 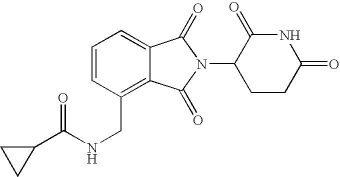
or a pharmaceutically acceptable salt, solvate or stereoisomer thereof.

4-[(Cyclopropanecarbonylamino)methyl]-2-(2,6-dioxopiperidin-3-yl)isoindole-1,3-dione can be prepared according to methods disclosed in U.S. Patent Application Publication No. 2003/0096841,which is incorporated herein by reference.

In one embodiment, 4-[(cyclopropanecarbonylamino)methyl]-2-(2,6-dioxopiperidin-3-yl)isoindole-1,3-dione is enantiomerically pure. In a further embodiment, 4-[(cyclopropanecarbonylamino)methyl]-2-(2,6-dioxopiperidin-3-yl)isoindole-1,3-dione is the (+)-enantiomer. In a further embodiment, 4-[(cyclopropanecarbonylamino)methyl]-2-(2,6-dioxopiperidin-3-yl)isoindole-1,3-dione is the (−)-enantiomer. In a further embodiment, 4-[(cyclopropanecarbonylamino)methyl]-2-(2,6-dioxopiperidin-3-yl)isoindole-1,3-dione is a racemic mixture.

### 5.4 Pharmaceutical Compositions and Dosage Forms

Pharmaceutical compositions can be used in the preparation of individual, single unit dosage forms. Pharmaceutical compositions and dosage forms of the invention comprise 4-[(cyclopropanecarbonylamino)methyl]-2-(2,6-dioxopiperidin-3-yl)isoindole-1,3-dione or a pharmaceutically acceptable salt, solvate or stereoisomer thereof and a second active agent. Pharmaceutical compositions and dosage forms of the invention can further comprise one or more carriers, excipients or diluents.

Pharmaceutical compositions and dosage forms of the invention can also comprise one or more additional active agents or ingredients. Consequently, pharmaceutical compositions and dosage forms of the invention may comprise 4-[(cyclopropanecarbonylamino)methyl]-2-(2,6-dioxopiperidin-3-yl)isoindole-1,3-dione and at least a second active agent. Examples of the optional second active agents are disclosed herein.

Single unit dosage forms of the invention are suitable for oral, mucosal (e.g., sublingual, nasal, vaginal, cystic, rectal, preputial, ocular, buccal or aural), parenteral (e.g., subcutaneous, intravenous, bolus injection, intramuscular or intraarterial), topical (e.g., eye drops or other ophthalmic preparations), transdermal or transcutaneous administration to a patient. Non-limiting examples of dosage forms include tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions or a water-in-oil liquid emulsions), solutions and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy and non-limiting examples of suitable excipients are provided herein. Whether a particular excipient is suitable for incorporation into a pharmaceutical composition or dosage form depends on a variety of factors well known in the art including, but not limited to, the way in -which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients can be accelerated by some excipients such as lactose or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler and a lubricant in pharmaceutically compatible and pharmaceutically acceptable amounts. Particular lactose-free dosage forms comprise active ingredients, microcrystalline cellulose, pre-gelatinized starch and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Non-limiting examples of suitable packaging include hermetically sealed foils, plastics, unit dose containers (e.g., vials), blister packs and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers or salt buffers. Like the amounts and types of excipients, the amounts and specific types of active ingredients in a dosage form may differ depending on factors such as, but not limited to, the route by which it is to be administered to patients. However, typical dosage forms of the invention comprise 4-[(cyclopropanecarbonylamino)methyl]-2-(2,6-dioxopiperidin-3-yl)isoindole-1,3-dione or a pharmaceutically acceptable salt, solvate or stereoisomer thereof in an amount of from about 0.10 to about 150 mg. Typical dosage forms comprise 4-[(cyclopropanecarbonylamino)methyl]-2-(2,6-dioxopiperidin-3-yl)isoindole-1,3-dione or a pharmaceutically acceptable salt, solvate or stereoisomer thereof in an amount of about 0.1, 1, 2, 5, 7.5, 10, 12.5, 15, 17.5, 20, 25, 50, 100, 150 or 200 mg. In a particular embodiment, a dosage form comprises 4-(amino)-2-(2,6-dioxo(3-piperidyl))-isoindoline-1,3-dione in an amount of about 1, 2, 5, 10, 25 or 50 mg. In a specific embodiment, a dosage form comprises excipients in an amount of about 5, 10, 25 or 50 mg. Typical dosage forms comprise the second active ingredient in an amount of 1 to about 1000 mg, from about 5 to about 500 mg, from about 10 to about 350 mg or from about 50 to about 200 mg. Of course, the specific amount of the agent will depend on the specific agent used, the type of disease or disorder being treated or managed and the amount(s) of 4-[(cyclopropanecarbonylamino)methyl]-2-(2,6-dioxopiperidin-3-yl)isoindole-1,3-dione and any optional additional second active agents concurrently administered to the patient.

### 5.4.1 Oral Dosage Forms

Pharmaceutical compositions of the invention that are suitable for oral administration can be presented as discrete dosage forms, such as, but are not limited to, tablets (e.g., chewable tablets), caplets, capsules and liquids (e.g., flavored syrups). Such dosage forms contain predetermined amounts of active ingredients and can be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. Non-limiting examples of excipients suitable for use in oral liquid or aerosol dosage forms include water, glycols, oils, alcohols, flavoring agents, preservatives and coloring agents. Non-limiting examples of excipients suitable for use in solid oral dosage forms (e.g., powders, tablets, capsules and caplets) include starches, sugars, micro-crystalline cellulose, diluents, granulating agents, lubricants, binders and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers or both and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Non-limiting examples of excipients that can be used in oral dosage forms of the invention include binders, fillers, disintegrants and lubricants. Non-limiting examples of binders suitable for use in pharmaceutical compositions and dosage forms include corn starch, potato starch or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose and mixtures thereof.

Non-limiting examples of suitable forms of microcrystalline cellulose include the materials sold as AVICEL-PH-101, AVICEL-PH-103 AVICEL RC-581, AVICEL-PH-105 (available from FMC Corporation, American Viscose Division, Avicel Sales, Marcus Hook, Pa.) and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Non-limiting examples of fillers suitable for use in the pharmaceutical compositions and dosage forms disclosed herein include talc, calcium carbonate (e.g., granules or powder), microcrystalline cellulose, powdered cellulose, dextrates, kaolin, mannitol, silicic acid, sorbitol, starch, pre-gelatinized starch and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation and is readily discernible to those of ordinary skill in the art. Typical pharmaceutical compositions comprise from about 0.5 to about 15 weight percent of disintegrant, preferably from about 1 to about 5 weight percent of disintegrant.

Non-limiting examples of disintegrants that can be used in pharmaceutical compositions and dosage forms of the invention include agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums and mixtures thereof.

Non-limiting examples of lubricants that can be used in pharmaceutical compositions and dosage forms of the invention include calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laureate, agar and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.) and mixtures thereof. If used at all, lubricants are typically used in an amount of less than about 1 weight percent of the pharmaceutical compositions or dosage forms into which they are incorporated.

A particular solid oral dosage form of the invention comprises 4-[(cyclopropanecarbonylamino)methyl]-2-(2,6-dioxopiperidin-3-yl)isoindole-1,3-dione, anhydrous lactose, microcrystalline cellulose, polyvinylpyrrolidone, stearic acid, colloidal anhydrous silica and gelatin.

### 5.4.2 Delayed Release Dosage Forms

Active ingredients of the invention can be administered by controlled release means or by delivery devices that are well known to those of ordinary skill in the art. Non-limiting examples of controlled release means or delivery devices include those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556 and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency and increased patient compliance. In addition, controlled-release formulations can be used to affect the time of onset of action or other characteristics, such as blood levels of the drug and can thus affect the occurrence of side (e.g., adverse) effects.

Most controlled-release formulations are designed to initially release an amount of drug (active ingredient) that promptly produces the desired therapeutic effect and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water or other physiological conditions or compounds.

### 5.4.3 Parental Dosage Forms

Parenteral dosage forms can be administered to patients by various routes including, but not limited to, subcutaneous, intravenous (including bolus injection), intramuscular and intraarterial. Because their administration typically bypasses patients' natural defenses against contaminants, parenteral dosage forms are preferably sterile or capable of being sterilized prior to administration to a patient. Non-limiting examples of parenteral dosage forms include solutions ready for injection, dry products ready to be dissolved or suspended in a pharmaceutically acceptable vehicle for injection, suspensions ready for injection and emulsions.

Suitable vehicles that can be used to provide parenteral dosage forms of the invention are well known to those skilled in the art. Non-limiting examples of suitable vehicles include Water for Injection USP; aqueous vehicles such as, but not limited to, Sodium Chloride Injection, Ringer's Injection, Dextrose Injection, Dextrose and Sodium Chloride Injection and Lactated Ringer's Injection; water-miscible vehicles such as, but not limited to, ethyl alcohol, polyethylene glycol and polypropylene glycol; and non-aqueous vehicles such as, but not limited to, corn oil, cottonseed oil, peanut oil, sesame oil, ethyl oleate, isopropyl myristate and benzyl benzoate.

Compounds that increase the solubility of one or more of the active ingredients disclosed herein can also be incorporated into the parenteral dosage forms of the invention. For example, cyclodextrin and its derivatives can be used to increase the solubility of 4-[(cyclopropanecarbonylamino)methyl]-2-(2,6-dioxopiperidin-3-yl)isoindole-1,3-dione and its derivatives.

### 5.4.4 Topical and Mucosal Dosage Forms

Drugs can be applied locally to the skin and its adnexa or to a variety of mucous membranes. The routes that can be used include sublingual, nasal, vaginal, cystic, rectal, preputial, ocular, buccal or aural. Many dosage forms have been developed to deliver active principles to the site of application to produce local effects. Non-limiting examples of topical and mucosal dosage forms of the invention include ointments, creams, gels, pastes, dusting powders, lotions, sprays, liniments, poultices, aerosols, solutions, emulsions, suspensions, eye drops or other ophthalmic preparations or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed by this invention are well known to those skilled in the pharmaceutical arts and depend on the particular tissue to which a given pharmaceutical composition or dosage form will be applied. Non-limiting examples of typical excipients include water, acetone, ethanol, ethylene glycol, propylene glycol, butane-1,3-diol, isopropyl myristate, isopropyl palmitate, mineral oil and mixtures thereof to form solutions, emulsions or gels, which are non-toxic and pharmaceutically acceptable.

Moisturizers such as occlusives, humectants, emollients and protein rejuvenators can also be added to pharmaceutical compositions and dosage forms if desired. Examples of such additional ingredients are well known in the art. See, e.g., Remington's Pharmaceutical Sciences, 16^{th} and 18^{th} eds., Mack Publishing, Easton Pa. (1980 & 1990).

Occlusives are substances that physically block water loss in the stratum corneum. Non-limiting examples of occlusives include petrolatum, lanolin, mineral oil, silicones such as dimethicone, zinc oxide and combinations thereof. Preferably, the occlusives are petrolatum and lanolin, more preferably petrolatum in a minimum concentration of 5%.

Humectants are substances that attract water when applied to the skin and theoretically improve hydration of the stratum corneum. However, the water that is drawn to the skin is water from other cells, not atmospheric water. With this type of moisturizer, evaporation from the skin can continue and actually can make the dryness worse. Non-limiting examples of humectants include glycerin, sorbitol, urea, alpha hydroxy acids, sugars and combinations thereof. Preferably, the humectants are alpha hydroxy acids, such as glycolic acid, lactic acid, malic acid, citric acid and tartaric acid.

Emollients are substances that smooth skin by filling spaces between skin flakes with droplets of oil, and are not usually occlusive unless applied heavily. When combined with an emulsifier, they may help hold oil and water in the stratum corneum. Vitamin E is a common additive, which appears to have no effect, except as an emollient. Likewise, other vitamins, for example, A and D, are also added, but their effect is questionable. Non-limiting examples of emollients include mineral oil, lanolin, fatty acids, cholesterol, squalene, structural lipids and combinations thereof.

Protein rejuvenators are substances that rejuvenate the skin by replenishing essential proteins. Non-limiting examples of protein rejuvenators include collagen, keratin, elastin and combinations thereof.

The pH of a pharmaceutical composition or dosage form may also be adjusted to improve delivery of one or more active ingredients. Similarly, the polarity of a solvent carrier, its ionic strength or tonicity can be adjusted to improve delivery. For example, absorption through the skin can also be enhanced by occlusive dressings, inunction or the use of dimethyl sulfoxide as a carrier. Compounds such as metal stearates (e.g., calcium stearate, zinc stearate, magnesium stearate, sodium stearate, lithium stearate, potassium stearate, etc.) can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant and as a delivery-enhancing or penetration-enhancing agent. Different salts, hydrates or solvates of the active ingredients can be used to further adjust the properties of the resulting composition.

### 5.5 Dosages

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically sufficient dose can be estimated initially from cell culture assays. A dose can be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

In one embodiment of the invention, 4-[(cyclopropanecarbonylamino)methyl]-2-(2,6-dioxopiperidin-3-yl)isoindole-1,3-dione can be administered orally and in single or divided daily doses in an amount of from about 0.10 to about 150 mg/day. In a particular embodiment, 4-[(cyclopropanecarbonylamino)methyl]-2-(2,6-dioxopiperidin-3-yl)isoindole-1,3-dione can be administered in an amount of from about 0.1 to about 1 mg per day or alternatively from about 0.1 to about 5 mg every other day. In another embodiment, 4-[(cyclopropanecarbonylamino)methyl]-2-(2,6-dioxopiperidin-3-yl)isoindole-1,3-dione can be administered in an amount of from about 1 to about 25 mg per day or alternatively from about 10 to about 50 mg every other day. In a certain embodiment, 4-[(cyclopropanecarbonylamino)methyl]-2-(2,6-dioxopiperidin-3-yl)isoindole-1,3-dione can be administered in an amount of about 50 mg per day. In another embodiment, 4-[(cyclopropanecarbonylamino)methyl]-2-(2,6-dioxopiperidin-3-yl)isoindole-1,3-dione can be administered in an amount of about 25 mg per day. In another embodiment, 4-[(cyclopropanecarbonylamino)methyl]-2-(2,6-dioxopiperidin-3-yl)isoindole-1,3-dione can be administered in an amount of about 10 mg per day.

The amount of the pharmaceutical composition administered according to the methods of the invention will depend on the subject being treated, the severity of the disorder or symptom of the disorder, the manner of administration, the frequency of administration and the judgement of the prescribing physician.

The frequency of administration is in the range of about an hourly dose to a monthly dose. In specific embodiments, administration is from 8 times per day to once every other day or from 1 to 3 times per day. In a specific embodiment, a pharmaceutical composition of the invention is administered chronically, e.g., daily.

### 5.6 Routes of Administration

The component or components of a therapeutic composition of the invention can be introduced parenterally, topically or transmucosally, e.g., nasally, orally, rectally or transdermally. In some embodiments, administration is parenteral, e.g., via intravenous injection and also including, but not limited to, intra-arteriole, intramuscular, intradermal, subcutaneous, intraperitoneal, intraventricular and intracranial administration. In certain embodiments, the component or components of a therapeutic composition of the invention is introduced orally, nasally or via inhalation or insufflation. In othe embodiments, the component or components of a therapeutic composition of the invention is introduced topically in a form including ointments, creams, gels, pastes, dusting powders, lotions, sprays, liniments, poultices, aerosols, solutions, emulsions and suspensions.

As used herein, administration of components of a therapeutic composition can mean either concurrent or sequential administration of each component.

In one embodiment of the invention, 4-[(cyclopropanecarbonylamino)methyl]-2-(2,6-dioxopiperidin-3-yl)isoindole-1,3-dione can be administered topically. In a further embodiment of the invention, 4-[(cyclopropanecarbonylamino)methyl]-2-(2,6-dioxopiperidin-3-yl)isoindole-1,3-dione can be administered topically in a dosage form selected from the group consisting of ointments, creams, gels, pastes, dusting powders, lotions, sprays, liniments, poultices, aerosols, solutions, emulsions and suspensions.

### 6. EXAMPLES

Some embodiments of the invention are illustrated by the following non-limiting examples. The examples should not be construed as a limitation in the scope thereof. The scope of the invention is defined solely by the appended claims.

### Example 1

### Preparation of 4-[(Cyclopropanecarbonylamino)methyl]-2-(2,6-dioxopiperidin-3-yl)isoindole-1,3-dione

Step 1—Preparation of 4-[(N,N-Dimethylhydrazono)methyl]isobenzofuran-1,3-dione. Maleic anhydride (2) (277.5 g, 2.83 moles, from Aldrich Chemicals, Milwaukee, Wis.) and ethyl acetate (1050 ml) were charged into a 5 L three-necked flask at room temperature under nitrogen. A solution of 2-furaldehyde N,N-dimethylhydrazone (300 g, 2.2 moles, from Aldrich Chemicals, Milwaukee, Wis.) in ethyl acetate (450 ml) was charged into the flask. After the reaction mixture was stirred for 5-10 minutes, trifluoroacetic acid (12.4 g, 0.11 mole, 5 mol %, from Aldrich Chemicals, Milwaukee, Wis.) was charged into the flask over 15-20 minutes. A latent exotherm (˜15-25° C. above room temperature) was observed. After the exotherm had subsided, the reaction mixture was heated to 45-55° C. for 6 hours, or alternatively, the reaction mixture was stirred for 8 hours at room temperature. At end of the respective reaction period (8 hours for room temperature reaction or 6 hours for the heated reaction), the reaction mixture was cooled to room temperature if necessary. After the reaction mixture was filtered at room temperature under vacuum, the filtered solid was washed sequentially with 600 ml of ethyl acetate, 2.4 L of deionized water, and 600 ml of heptane. The solid was dried in a tray at 55-60° C. under vacuum for 8-12 hours. The yield of 4-[(N,N-dimethylhydrazono)methyl]isobenzofuran-1,3-dione was found to be 400 g (84%).

Step 2—Preparation of 4-[(N,N-Dimethylhydrazono)methyl]-2-(2,6-dioxopiperidin-3-yl)isoindole-1,3-dione. 4-[(N,N-Dimethylhydrazono)methyl]isobenzofuran-1,3-dione (300 g, 1.38 moles, prepared previously) was charged into a 5 L three-necked flask, followed by the addition of α-amino glutarimide hydrochloride (189 g, 1.15 mol, from Evotec OAI, Oxfordshire, UK), imidazole (780 g, 11.5 mol, from Aldrich Chemicals, Milwaukee, Wis.) and acetonitrile (2.28 L, from Fisher Scientific, Pittsburgh, Pa.), at room temperature under nitrogen to form a solution. After acetic acid (688 g, 11.5 mol, from Fisher Scientific, Pittsburgh, Pa.) was charged into the solution at room temperature, the reaction mixture was stirred for 10-15 minutes. An exotherm (˜10-15° C. above room temperature) was observed. After the exotherm had subsided, the reaction mixture was heated to 75-82° C. for 2-3 hours while the H₂O formed during the reaction was removed by distilling out 378 ml of an acetonitrile/water azeotrope. Next, the reaction mixture was cooled to 65° C. and diluted with water (756 ml) while it was stirred at room temperature. The reaction mixture was filtered under vacuum and the filtered solid was washed sequentially with deionized water (1512 ml) and heptane (378 ml). The solid was dried in a tray at 55-60° C. under vacuum for 8-12 hours. The yield of 4-[(N,N-dimethylhydrazono)methyl]-2-(2,6-dioxopiperidin-3-yl)isoindole-1,3-dione was found to be 311 g (83%).

Step 3—Preparation of 4-Aminomethyl-2-(2,6-dioxopiperidin-3-yl)isoindole-1,3-dione hydrochloride salt. 4-[(N,N-Dimethylhydrazono)methyl]-2-(2,6-dioxopiperidin-3-yl)isoindole-1,3-dione (100 g, 0.304 mol, prepared previously) was charged into a 5 L Parr-vessel, followed by the addition of 10% Pd/C (50% wet, 4 g, 4 wt %, from Johnson Matthey, London, UK), a mixture of methanol and water in a volume ratio of 2:1 (1500 ml), and methanesulfonic acid (58.5 g, 0.609 mol, from Aldrich Chemicals, Milwaukee, Wis.) at room temperature under nitrogen. The reaction mixture was purged with sequentially with nitrogen (3 times) and hydrogen (3 times). The reaction mixture was stirred at room temperature over 18-20 hours with hydrogen maintained at a pressure between 40-50 psi. Alternatively, the reaction mixture was stirred at 40° C. over 6-8 hours with hydrogen maintained at a pressure between 40-50 psi. Next, the reaction was filtered through a celite bed (1 inch thickness) and the celite bed was washed with a mixture of methanol and water in a volume ratio of 2:1 (200 ml). The reaction mixture was cooled to room temperature if necessary and then filtered. The filtrate was concentrated under reduced pressure (15-20 torr) at 35-45 ° C. until 1.36 L (80%) of the methanol and water mixture was collected. After the concentrated filtrate was diluted with acetone (500 ml) and cool in an ice-bath at 0-5° C., 12N hydrochloric acid (102 ml, 1.22 mol) was added at a rate such that the reaction temperature was maintained between 0 and 5° C. Next, the acetone solution was warmed to room temperature. When turbidity was observed in the acetome solution, 2 g (2 wt. %) of 4-[(N,N-dimethylhydrazono)methyl]-2-(2,6-dioxopiperidin-3-yl)isoindole-1,3-dione was added. The mixture was stirred at room temperature for 15 hours while 4-[(N,N-dimethylhydrazono)methyl]-2-(2,6-dioxopiperidin-3-yl)isoindole-1,3-dione precipitated out from the acetone solution. The mixture was charged with ethyl acetate (300 ml) and stirred for a further 2 hours at room temperature. The mixture was filtered and washed sequentially with acetonitrile (100 ml), ethyl acetate (100 ml) and heptane (100 ml). The filtered solid was dried in a tray at 55-60° C. under vacuum for 12 hours. The yield of 4-aminomethyl-2-(2,6-dioxopiperidin-3-yl)isoindole-1,3-dione hydrochloride salt was found to be 77 g (78%).

Step 4—Preparation of 4-[(Cyclopropanecarbonylamino)methyl]-2-(2,6-dioxopiperidin-3-yl)isoindole-1,3-dione Hydrochloride Salt. After 4-Aminomethyl-2-(2,6-dioxopiperidin-3-yl)isoindole-1,3-dione hydrochloride salt (100.0 g, 0.31 moles, prepared previously) and acetonitrile (1.0 L) were charged into a 5 L three-necked flask, the reaction mixture was cooled to 0-5° C. Next, cyclopropanecarbonyl chloride (35.5 g, 30.8 ml, 0.34 mole, from Aldrich Chemicals, Milwaukee, Wis.) was added to the cooled reaction mixture over 20-30 minutes at 0-5° C. with stirring. N,N-diisopropylethylamine (79.9 g, 107.7 ml, 0.62 mole, from Aldrich Chemicals, Milwaukee, Wis.) was added to the reaction mixture over 45-60 minutes while the temperature was maintained at 0-5° C. The reaction mixture was warmed to 18-22° C. and stirred for 3 additional hours until the reaction was complete. After the reaction mixture was cooled to 0-5° C., 2N aqueous hydrochloric acid (1.0 L) was added over 20-30 minutes while the temperature was maintained at 0-5° C. The reaction mixture was stirred for 1 hour while the reaction mixture gradually increased to 18-22° C. A white solid precipitated and was filtered out under vacuum and washed with 1.0 L deionized water. The white solid was dried in a tray at 50-55° C. under a pressure of 100-125 mm of Hg. The yield of 4-[(cyclopropanecarbonylamino)methyl]-2-(2,6-dioxopiperidin-3-yl)isoindole-1,3-dione hydrochloride salt was found to be 100.95 g (92%).

If a racemic compound of 4-[(cyclopropanecarbonylamino)methyl]-2-(2,6-dioxopiperidin-3-yl)isoindole-1,3-dione is desired, a racemic a-amino glutarimide hydrochloride can be used in step 2. Conversely, if an enantiomerically pure compound of Formula (I) is desired, an enantiomerically pure α-amino glutarimide hydrochloride can be used in step 2. Alternatively, if an enantiomerically pure compound of 4-[(cyclopropanecarbonylamino)methyl]-2-(2,6-dioxopiperidin-3-yl)isoindole-1,3-dione is desired, a racemic mixture of 4-[(cyclopropanecarbonylamino)methyl]-2-(2,6-dioxopiperidin-3-yl)isoindole-1,3-dione can be prepared and then the racemic mixture can be resolved into the enantiomers by conventional resolution techniques such as biological resolution and chemical resolution. In general, biological resolution uses a microbe which metabolizes one specific enantiomer leaving the other alone. In chemical resolution, the racemic mixture is converted into two diastereoisomers that can be separated by conventional techniques such as fractional crystallization and chromatographies. Once separated, the diasteriosomeric forms are converted separately back to the enantiomers.

The active ingredient in Examples 2-11 below is 4-[(cyclopropanecarbonylamino)methyl]-2-(2,6-dioxopiperidin-3-yl)isoindole-1,3-dione or a pharmaceutically acceptable salt, solvate or stereoisomer thereof.

### Example 2

Tablets, each containing 50 milligrams of an active ingredient, can be prepared in the following manner:

Composition (for 1000 tablets)

| | | |
|---|---|---|
| active ingredient | 50.0 | grams |
| lactose | 50.7 | grams |
| wheat starch | 7.5 | grams |
| polyethylene glycol 6000 | 5.0 | grams |
| talc | 5.0 | grams |
| magnesium stearate | 1.8 | grams |

| | |
|---|---|
| demineralized water | q.s. |

The solid ingredients are first forced through a sieve of 0.6 mm mesh width. The active ingredient, the lactose, the talc, the magnesium stearate and half of the starch then are mixed. As mentioned earlier, the active ingredient is 4-[(cyclopropanecarbonylamino)methyl]-2-(2,6-dioxopiperidin-3-yl)isoindole-1,3-dione or a pharmaceutically acceptable salt, solvate or stereoisomer thereof. The other half of the starch is suspended in 40 milliliters of water and this suspension is added to a boiling solution of the polyethylene glycol in 100 milliliters of water. The resulting paste is added to the pulverulent substances and the mixture is granulated, if necessary with the addition of water. The granulate is dried overnight at 35° C., forced through a sieve of 1.2 mm mesh width and compressed to form tablets of approximately 6 mm diameter which are concave on both sides.

### Example 3

Tablets, each containing 100 milligrams of active ingredient, can be prepared in the following manner:

Composition (for 1000 tablets)

| | |
|---|---|
| active ingredient | 100.0 grams |
| lactose | 100.0 grams |
| wheat starch | 47.0 grams |
| magnesium stearate | 3.0 grams |

All the solid ingredients are first forced through a sieve of 0.6 mm mesh width. The active ingredient, the lactose, the magnesium stearate and half of the starch then are mixed. The other half of the starch is suspended in 40 milliliters of water and this suspension is added to 100 milliliters of boiling water. The resulting paste is added to the pulverulent substances and the mixture is granulated, if necessary with the addition of water. The granulate is dried overnight at 35° C., forced through a sieve of 1.2 mm mesh width and compressed to form tablets of approximately 6 mm diameter which are concave on both sides.

### Example 4

Tablets for chewing, each containing 75 milligrams of active ingredient, can be prepared in the following manner:

Composition (for 1000 tablets)

| | | |
|---|---|---|
| active ingredient | 75.0 | grams |
| mannitol | 230.0 | grams |
| lactose | 150.0 | grams |
| talc | 21.0 | grams |
| glycine | 12.5 | grams |
| stearic acid | 10.0 | grams |
| saccharin | 1.5 | grams |

| | |
|---|---|
| 5% gelatin solution | q.s. |

All the solid ingredients are first forced through a sieve of 0.25 mm mesh width. The mannitol and the lactose are mixed, granulated with the addition of gelatin solution, forced through a sieve of 2 mm mesh width, dried at 50° C. and again forced through a sieve of 1.7 mm mesh width. The active ingredient, the glycine and the saccharin are carefully mixed. The mannitol, the lactose granulate, the stearic acid and the talc are added and the whole is mixed thoroughly and compressed to form tablets of approximately 10 mm diameter which are concave on both sides and have a breaking groove on the upper side.

### Example 5

Tablets, each containing 10 milligrams of active ingredient, can be prepared in the following manner:

Composition (for 1000 tablets)

| | | |
|---|---|---|
| active ingredient | 10.0 | grams |
| lactose | 328.5 | grams |
| corn starch | 17.5 | grams |
| polyethylene glycol 6000 | 5.0 | grams |
| talc | 25.0 | grams |
| magnesium stearate | 4.0 | grams |

| | |
|---|---|
| demineralized water | q.s. |

The solid ingredients are first forced through a sieve of 0.6 mm mesh width. Then the active ingredient, lactose, talc, magnesium stearate and half of the starch are intimately mixed. The other half of the starch is suspended in 65 milliliters of water and this suspension is added to a boiling solution of the polyethylene glycol in 260 milliliters of water. The resulting paste is added to the pulverulent substances, and the whole is mixed and granulated, if necessary with the addition of water. The granulate is dried overnight at 35° C., forced through a sieve of 1.2 mm mesh width and compressed to form tablets of approximately 10 mm diameter which are concave on both sides and have a breaking notch on the upper side.

### Example 6

Gelatin dry-filled capsules, each containing 100 milligrams of active ingredient, can be prepared in the following manner:

Composition (for 1000 capsules)

| | |
|---|---|
| active ingredient | 100.0 grams |
| microcrystalline cellulose | 30.0 grams |
| sodium lauryl sulphate | 2.0 grams |
| magnesium stearate | 8.0 grams |

The sodium lauryl sulphate is sieved into the active ingredient through a sieve of 0.2 mm mesh width and the two components are intimately mixed for 10 minutes. The microcrystalline cellulose is then added through a sieve of 0.9 mm mesh width and the whole is again intimately mixed for 10 minutes. Finally, the magnesium stearate is added through a sieve of 0.8 mm width and, after mixing for a further 3 minutes, the mixture is introduced in portions of 140 milligrams each into size 0 (elongated) gelatin dry-fill capsules.

### Example 7

A 0.2% injection or infusion solution can be prepared, for example, in the following manner:

Composition

| | | |
|---|---|---|
| active ingredient | 5.0 | grams |
| sodium chloride | 22.5 | grams |
| phosphate buffer pH 7.4 | 300.00 | grams |
| demineralized water | to 2500.0 | milliliters |

The active ingredient is dissolved in 1000 milliliters of water and filtered through a microfilter. The buffer solution is added and the whole is made up to 2500 milliliters with water. To prepare dosage unit forms, portions of 1.0 or 2.5 milliliters each are introduced into glass ampoules (each containing respectively 2.0 or 5.0 milligrams of active ingredient).

### Example 8

An ointment for topical use can be prepared, for example, in the following manner:

Composition

| | | |
|---|---|---|
| active ingredient | 10 | g |
| petrolatum | 80 | g |
| mineral oil | 120 | g |
| 2% saline solution | 2 | L |
| triamcinolone acetonide | 0.5 | g |

The above ingredients are mixed uniformly to form an ointment using a conventional mixer or homogenizer, by shaking or by ultrasonic energy.

### Example 9

A gel for topical use can be prepared, for example, in the following manner:

Composition

| | | |
|---|---|---|
| active ingredient | 10 | g |
| Carboxylmethyl cellulose | 0.2 | g |
| Glycerin | 40.0 | g |
| 0.4 mole/L Citrate buffer | 25.0 | g |
| Distilled water | to 100 | g |

The above ingredients are mixed uniformly to form a gel using a conventional mixer or homogenizer, by shaking or by ultrasonic energy.

### Example 10

A paste for topical use can be prepared, for example, in the following manner:

Composition

| | | |
|---|---|---|
| active ingredient | 10 | g |
| Carboxymethyl cellulose | 2.0 | g |
| Glycerin | 25.0 | g |
| Cetanol | 2.8 | g |
| Glyceryl monostearate | 9.3 | g |
| Tween 80 | 2.0 | g |
| Glucuronic acid | 1.0 | g |
| 0.4 mole/l Citrate buffer | 20.0 | g |
| Distilled water | to 100 | g |

The above ingredients are mixed uniformly to form a paste using a conventional mixer or homogenizer, by shaking or by ultrasonic energy.

### Example 11

A liquid composition for topical use can be prepared, for example, in the following manner:

Composition

| | | |
|---|---|---|
| active ingredient | 10 | g |
| Carboxymethyl cellulose | 0.1 | g |
| Glycerin | 15.0 | g |
| 0.4 mole/l Citrate buffer (pH 4.5) | 50.0 | g |
| Distilled water | to 100 | g |

The solid ingredients are dispersed/dissolved in the liquid ingredients uniformly to form a liquid using a conventional mixer or homogenizer, by shaking or by ultrasonic energy.

### Example 12

A spray for topical use can be prepared, for example, in the following manner:

Composition

| | |
|---|---|
| The liquid composition of Example 11 | 100.0 g |
| Freon 114 | 100.0 g |

The liquid composition and Freon 114 are filled into Teflon-coated aluminum spray containers.

All of the references cited herein are incorporated by reference in their entirety. While the invention has been described with respect to the particular embodiments, it will be apparent to those skilled in the art that various changes and modifications can be made without departing from the spirit and scope of the invention as recited by the appended claims.

The embodiments of the invention described above are intended to be merely exemplary and those skilled in the art will recognize or will be able to ascertain using no more than routine experimentation, numerous equivalents of specific compounds, materials and procedures. All such equivalents are considered to be within the scope of the invention and are encompassed by the appended claims.

What is claimed is:

1. A method of treating, managing or preventing cutaneous lupus, which comprises administering to a patient in need of such treatment, management or prevention a therapeutically or prophylactically effective amount of 4-[(cyclopropanecarbonylamino)methyl]-2-(2,6-dioxopiperidin-3-yl)isoindole-1,3-dione or a pharmaceutically acceptable salt, solvate or stereoisomer thereof.

2. The method of claim 1, wherein the 4-[(cyclopropanecarbonylamino)methyl]-2-(2,6-dioxopiperidin-3-yl)isoindole-1,3-dione is a racemic mixture.

3. The method of claim 1, wherein the 4-[(cyclopropanecarbonylamino)methyl]-2-(2,6-dioxopiperidin-3-yl)isoindole-1,3-dione is the (+)-enantiomer substantially free of its counter enantiomer.

4. The method of claim 1, wherein the 4-[(cyclopropanecarbonylamino)methyl]-2-(2,6-dioxopiperidin-3-yl)isoindole-1,3-dione is the (−)-enantiomer substantially free of its counter enantiomer.

5. The method of claim 1, wherein the 4-[(cyclopropanecarbonylamino)methyl]-2-(2,6-dioxopiperidin-3-yl)isoindole-1,3-dione is a pharmaceutically acceptable salt.

6. The method of claim 1, wherein the 4-[(cyclopropanecarbonylamino)methyl]-2-(2,6-dioxopiperidin-3-yl)isoindole-1,3-dione is a pharmaceutically acceptable solvate.

7. The method of claim 1, wherein the 4-[(cyclopropanecarbonylamino)methyl]-2-(2,6-dioxopiperidin-3-yl)isoindole-1,3-dione is a pharmaceutically acceptable stereoisomer.

8. The method of claim 1, wherein the cutaneous lupus is acute cutaneous lupus erythematosus

9. The method of claim 1, wherein the cutaneous lupus is subacute cutaneous lupus erythematosus.

10. The method of claim 1, wherein the cutaneous lupus is discoid lupus erythematosus.

11. The method of claim 1, wherein the cutaneous lupus is neonatal lupus erythematosus.

12. The method of claim 1, wherein the cutaneous lupus is lupus erythematosus of childhood.

13. The method of claim 1 further comprising administering to the patient therapeutically or prophylactically effective amount of at least a second active agent.

14. The method of claim 13, wherein the second active agent is an anti-inflammatory, an immunomodulatory compound, an anti-malarial, an immunosuppressant, an antibiotic, an antiviral, an immunoglobulin, an immunologic-enhancing drug or a hormone.

15. The method of claim 14, wherein the anti-inflammatory is acetaminophen, a 5-aminosalicylic acid derivative, a salicylate, a corticosteroid or a nonsteroidal anti-inflammatory drug.

16. The method of claim 14, wherein the immunomodulatory compound is thalidomide, azathioprine, cyclophosphamide, methotrexate or cyclosporin.

17. The method of claim 14, wherein the antibiotic is ampicillin, tetracycline, penicillin, cephalosporin, streptomycin, kanamycin or erythromycin

18. The method of claim 14, wherein the antiviral is amantadine, rimantadine, acyclovir or ribavirin.

19. The method of claim 14, wherein the immunologic-enhancing drug is levamisole or isoprinosine.

20. The method of claim 14, wherein the biologic is a transfer factor, an interleukin or an interferon.

21. The method of claim 14, wherein the hormone is thymic hormone.

22. The method of claim 1 wherein the 4-[(cyclopropanecarbonylamino)methyl]-2-(2,6-dioxopiperidin-3-yl)isoindole-1,3-dione or a pharmaceutically acceptable salt, solvate or stereoisomer thereof is administered orally.

23. The method of claim 1, wherein the 4-[(cyclopropanecarbonylamino)methyl]-2-(2,6-dioxopiperidin-3-yl)isoindole-1,3-dione or a pharmaceutically acceptable salt, solvate or stereoisomer thereof is administered parenterally.

24. The method of claim 1, wherein the 4-[(cyclopropanecarbonylamino)methyl]-2-(2,6-dioxopiperidin-3-yl)isoindole-1,3-dione or a pharmaceutically acceptable salt, solvate or stereoisomer thereof is administered topically.

25. The method of claim 24, wherein the 4-[(cyclopropanecarbonylamino)methyl]-2-(2,6-dioxopiperidin-3-yl)isoindole-1,3-dione or a pharmaceutically acceptable salt, solvate or stereoisomer thereof is administered topically in a dosage form selected from the group consisting of ointments, creams, gels, pastes, dusting powders, lotions, sprays, liniments, poultices, aerosols, solutions, emulsions, suspensions and combinations thereof.

26. A pharmaceutical composition comprising 4-[(cyclopropanecarbonylamino)methyl]-2-(2,6-dioxopiperidin-3-yl)isoindole-1,3-dione or a pharmaceutically acceptable salt, solvate or stereoisomer thereof in a therapeutically or prophylactically effective amount for treating, managing or preventing cutaneous lupus.

27. The pharmaceutical composition of claim 26 further comprising at least a second active agent wherein the second active agent is an immunomodulatory compound, an anti-inflammatory, an anti-malarial, an immunosuppressant, an antibiotic, an antiviral, an immunoglobulin, an immunologic-enhancing drug or a hormone.

28. The pharmaceutical composition of claim 26 further comprising at least an ingredient selected from the group consisting of excipients, moisturizers, carriers, diluents, metal stearates and combinations thereof.

29. The pharmaceutical composition of claim 26, which is in a single unit dosage form.

30. The pharmaceutical composition of claim 26, which is in a single unit dosage form suitable for oral administration

31. The pharmaceutical composition of claim 26, which is in a single unit dosage form suitable for parenteral administration.

32. The pharmaceutical composition of claim 26, which is in a single unit dosage form suitable for topical administration.

33. The pharmaceutical composition of claim 33, wherein the single unit dosage form is selected from the group consisting of ointments, creams, gels, pastes, dusting powders, lotions, sprays, liniments, poultices, aerosols, solutions, emulsions, suspensions and combinations thereof.

34. The pharmaceutical composition of claim 34, further comprising an ingredient selected from the group consisting of excipients, moisturizers, carriers, diluents, metal stearates and combinations thereof.
